(19) United States
(12) Patent Application Publication
(10) Pub. No.: US 2017/0258374 A1
(43) Pub. Date: Sep. 14, 2017

(54) SYSTEM AND METHOD FOR AUTOMATIC POSTURE CALIBRATION

(71) Applicant: Lumo BodyTech, Inc, Mountain View, CA (US)

(72) Inventors: Daniel Ly, Mountain View, CA (US); Andreas Martin Hauenstein, San Mateo, CA (US); Chung-Che Charles Wang, Mountain View, CA (US); Andrew Robert Chang, Sunnyvale, CA (US)

(21) Appl. No.: 15/454,514

(22) Filed: Mar. 9, 2017

Related U.S. Application Data
(60) Provisional application No. 62/305,883, filed on Mar. 9, 2016.

(51) Int. Cl. A61B 5/11; A61B 5/00

(52) U.S. Cl. CPC A61B 5/1116; A61B 5/1118; A61B 5/486; A61B 5/7267; A61B 5/6804; A61B 5/112; A61B 5/7455; A61B 5/7475; A61B 2560/0223

(57) ABSTRACT

A system and method for posture feedback can include collecting kinematic data by an activity monitoring device coupled to a user; calibrating the kinematic data to a base walking orientation of the activity monitoring device, which comprises: detecting a walking activity state through the kinematic data, and when a walking activity state is detected, generating the base walking orientation from kinematic data; setting a posture correction factor; measuring user posture with the calibrated kinematic data; triggering posture feedback based on the user posture adjusted by the posture correction factor.

### CROSS-REFERENCE TO RELATED APPLICATIONS

This Application claims the benefit of U.S. Provisional Application No. 62/305,883, filed on 9 Mar. 2016, which is incorporated in its entirety by this reference.

### TECHNICAL FIELD

This invention relates generally to the field of posture feedback devices and more specifically to a new and useful system and method for automatic posture calibration.

### BACKGROUND

There are several variations of fitness and activity tracking and coaching devices available on the market. These products usually involve a sensor attached or worn by a user. One application of such products can be posture or ergonomic coaching. However, a common problem is that the sensing device is often inconsistently attached to the user causing problems in providing accurate posture coaching. The sensing device often uses some form of calibration, but proper calibration is challenging and at times depends on user involvement following prescribed actions to calibrate, which may be cumbersome for the user and error prone. Even with calibration, the sensing device can fail to accurately represent posture, ergonomics, or other biomechanical aspects during the course of activity because of changes of the user or orientation of the sensing device. Thus, there is a need in the posture feedback device field to create a new and useful system and method for automatic posture calibration. This invention provides such a new and useful system and method.

### BRIEF DESCRIPTION OF THE FIGURES

FIG. 1 is a schematic of a system of a preferred embodiment

FIG. 2 is a schematic representation of an exemplary design for coupling to clothing;

FIG. 3 is a flowchart representation of a method of a preferred embodiment;

FIG. 4 is a schematic representation of different calibration states;

FIG. 5 is a schematic representation of an exemplary coordinate system;

FIGS. 6-8 are graphical plots of exemplary data showing pitch and yaw correction of kinematic data;

FIG. 9 is a schematic representation of handling multiple activity states;

FIG. 10 is a flowchart representation of processes for calibrating the kinematic data to a base walking orientation;

FIG. 11 is a schematic representation of generating a posture correction factor from a base orientation matrix and a target orientation matrix; and

FIG. 12 is a flowchart representation of method for manual calibration of a preferred embodiment.

### DESCRIPTION OF THE EMBODIMENTS

The following description of the embodiments of the invention is not intended to limit the invention to these embodiments but rather to enable a person skilled in the art to make and use this invention.

### 1. Overview

A system and method for automatic posture calibration of a preferred embodiment functions to use orientation calibration of a device during different activity states so as to assess the posture and biomechanics of a user. The system and method is preferably applied in the context of an activity monitoring device used in providing activity data and/or posture data. Raw sensor data collected by an activity monitoring device (e.g., raw accelerometer data) can depend on user posture and sensor placement on the body. The system and method utilizes an approach that generates a transform of the sensor data and calibrates an activity monitoring device that accounts for position and orientation when worn. This calibration process can happen in the background, without conscious user involvement—autocalibration. After calibration, the sensor data can be abstracted away from sensor placement on the body and instead reflect user posture.

More specifically, the system and method of a preferred embodiment function to monitor posture of a first activity state by indirectly referencing a calibrated orientation of a second activity state and associated offsets between the first activity state and the second activity. For example, one preferred implementation establishes a reference orientation when the user is walking and then assesses posture in another activity state (e.g., sitting) based on offsets between good sitting posture and walking posture. Walking in particular is an activity where a user has been discovered to exhibit substantially consistent posture. Additionally, walking is easily detectable and periodically performed over the course of a sustained period, which enables recalibration of reference orientation.

The system and method can offer a number of potential benefits. As one potential benefit, the system and method can be robust to perturbance and movement of the sensor during use. A sensor with an inertial measurement unit (IMU) is generally used in sensing, detecting, and monitoring posture. The sensor will generally be coupled to a user by attaching or adhering to a portion of the body or an article of clothing. The relative orientation of the sensor and the user is critical in understanding the biomechanics of a user. However, a sensor may move just through normal use or even be actively moved or adjusted by user. The autocalibration (and recalibration) capabilities of the system and method can address such changes in relative orientation. Additionally, user calibrated settings may extend across multiple sessions of use or even multiple devices. User-initiated calibration for each use may be avoided. For example, a user may be able to have accurate, customized posture monitoring across multiple uses even when the sensor is coupled to the user with different relative orientations.

Another potential benefit of the system and method can be improved accuracy. Some variations of the system and method can customize posture sensing and monitoring to target a particular user or class of user.

As another potential benefit, the system and method may enable calibration without directing the user through multiple calibration processes. The user can simply calibrate a targeted posture, and the system can automatically calibrate when the user walks.

Similarly, another potential benefit of the system of the method can be flexibility in how an activity monitoring device is mounted to a user. The system and method could support physical coupling of the activity monitoring device in a variety of body locations such as the upper chest, the back, the pelvic region, limbs, and/or any suitable body location. Additionally the general orientation of the activity monitoring device and the user could vary. For example, the activity monitoring device could be attached to face up, down, right, left, front, back and/or any suitable orientation.

As another potential benefit, the posture sensing and monitoring of the system and method may be extended to multiple activity states such that posture can be uniquely monitored for sitting, prolonged standing, walking, running, driving, and/or any suitable activity.

As another potential benefit, the system and method can support other usability features such as posture feedback silencing. In some cases, a user may want to temporarily suspend active feedback when a posture target is not being achieved. In one implementation, a silencing feature can be integrated into the system and method. Furthermore, the mechanism for enabling the silencing feature can be simplified to performing the same action as signaling a calibration event. For example, a single button used in a substantially similar way by the user may be used to both coach the system on what to consider good posture and to temporarily pause posture feedback.

The system and method can be used for a variety of use cases such as posture coaching, ergonomic coaching, sensing biomechanical properties of an activity like running or biking, and/or any suitable use case. Herein, the system and method are primarily described as being used for monitoring spinal posture of a user, but the system and method could alternatively be used for calibrating and monitoring the orientation of any suitable body part.

### 2. System

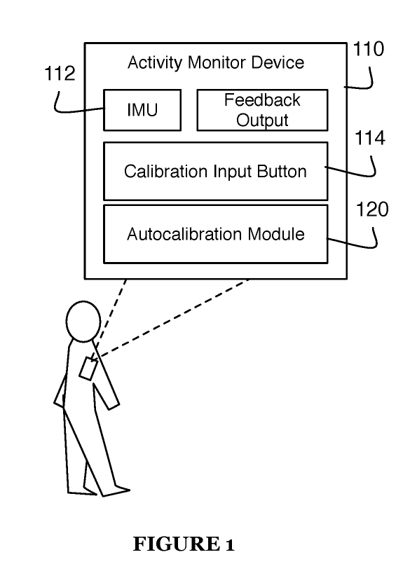

As shown in FIG. 1, a system for automatic posture calibration of a preferred embodiment includes an activity monitoring device 110 and an autocalibration module 120.

The activity monitoring device 110 of a preferred embodiment acts as a sensor for detecting movement and/or orientation of a user. The activity monitoring device 110 is preferably a wearable device that is coupled to a user. The activity monitoring device 110 can be directly worn or attached by a user or indirectly coupled by attaching to an article of worn clothing. In one variation, the activity monitoring device 110 is a standalone device that can operate independent of other components. In another variation, the activity monitoring device can be communicatively coupled to at least a second device such as an application operable on a personal computing device or a web service operable on a server system. A personal computing device can include a mobile phone, a smart watch, a smart wearable, and/or any suitable computing device. In one preferred embodiment, the activity monitoring device 110 includes a casing and/or fixture mechanism configured to removably attach to an article of clothing. The fastening mechanism could be a pin, a clip, or any suitable latching mechanism.

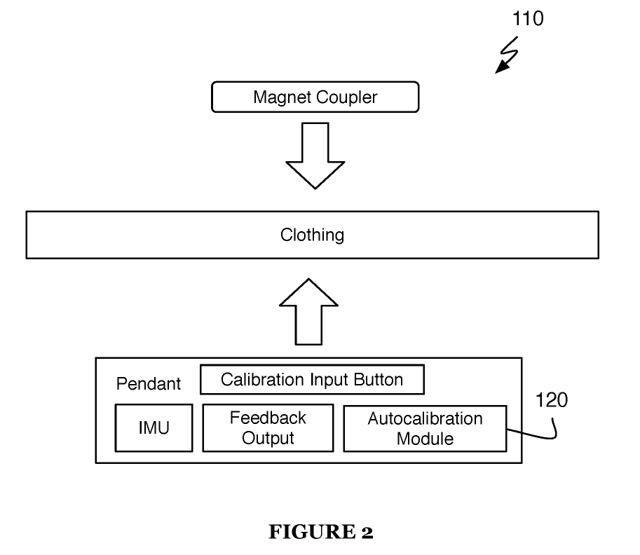

In a two-part pendant implementation shown in FIG. 2, the activity monitoring device 110 comprises a main housing (i.e., the “pendant”) and a magnet coupler. The pendant preferably houses the main computational components. The magnet coupler preferably magnetically couples to the pendant about a magnetic coupling region. At least one magnet can be positioned in the magnetic coupling region and/or the magnet coupler. The magnetic coupling is preferably sufficiently strong to promote attraction through a layer of clothing. A user can put the main housing on the underside of a garment, and then fix the pendant in place by magnetically coupling the magnet coupler on the opposite side of the garment. A button can be positioned below and surrounding the magnetic coupling region such that, a user can press on the magnet coupler to activate a button on the pendant.

The activity monitoring device 110 preferably includes a sensor system that includes an inertial measuring unit 112. The inertial measuring unit 112 functions to measure multiple kinematic properties of an activity. An inertial measurement unit 112 can include at least one accelerometer, gyroscope, magnetometer, and/or other suitable inertial sensors. The inertial measurement unit 112 preferably includes a set of sensors aligned for detection of kinematic properties along three orthogonal axes. In one variation, the inertial measurement unit 112 is a 9-axis motion-tracking device that includes a 3-axis gyroscope, a 3-axis accelerometer, and a 3-axis magnetometer. The activity monitoring device 110 can additionally include an integrated processor that provides sensor fusion. Sensor fusion can combine kinematic data from the various sensors to reduce uncertainty. In this application, it may be used to estimate orientation with respect to gravity and may be used in separating forces or sensed dynamics for data from a sensor. The on-device sensor fusion may provide other suitable sensor conveniences. Alternatively, multiple distinct sensors can be combined to provide a set of kinematic measurements. The on-device sensor fusion components may be controlled to calibrate the inertial measurement unit 112 according to the method described below.

A sensing system of the activity monitoring device 110 can additionally or alternatively include other sensors such as an altimeter, global positioning system (GPS), or any suitable sensor. Biometric sensors may additionally be included.

Additionally, the activity monitoring device 110 can include a communication channel to one or more computing devices or additional activity monitoring devices with one or more sensors. For example, an inertial measuring system can include a Bluetooth communication channel to a smart phone, and the smart phone can track and retrieve data on geolocation, distance covered, elevation changes, and other data.

The activity monitoring device no can additionally include a calibration input 114, which functions to enable a signal to be generated to trigger a calibration event used in directing calibration of the activity monitoring device no and/or signal any other suitable information. The calibration input 114 can be a physical or virtual button on the activity monitoring device such as the one described in the two-part pendant implementation above. The calibration input 114 could alternatively or additionally be a user input mechanism offered by a connected device such as a user application.

In one preferred operating state, activation of a calibration input triggers the collection of kinematic data used in determining a target (i.e., a reference) posture sample. For example, the user can direct the system on what is considered good posture by standing with good posture and then calibrating the system to recognize this posture by activating a calibration input and holding the posture for a minimum duration.

A calibration input 114 could be overloaded to direct other signals. For example, the calibration input 114 could additionally be configured to trigger a silencing event, which may temporarily suspend posture feedback. For example, the user may not want to be notified of his or her bad posture when they are reclining and relaxing. The activation of the calibration input, while in a particular bad posture, may be classified as a silencing event instead of a calibration event. In response, posture feedback can be paused until posture feedback is reactivated, after the user moves out of the “bad” posture for a minimum amount of time, or upon satisfying any suitable condition.

The activity monitor device 110 and/or another device of the system can include a user feedback mechanism, which can include at least one user interface element that can provide tactile feedback, audio feedback, graphical feedback (e.g., on device or in an application), informational feedback (e.g., data analysis representations), and/or other forms of feedback.

The autocalibration module 120 functions to process the kinematic data generated by the activity monitoring device. The autocalibration module 120 preferably includes operational logic configured to facilitate at least a portion of the calibration process described below. In particular, the autocalibration module 120 can be configured to calibrate kinematic data collected by the activity monitor to a base walking orientation. The autocalibration module 120 may additionally be configured to detect activity states, detect a calibration event, set a posture correction factor, and/or other processes in the method for automatic posture calibration. The autocalibration module 120 is preferably integrated into the activity monitoring device 110. Alternatively, part or the entire autocalibration module 120 can be integrated with a secondary device such as a smart phone or smart watch. For example, a user application may be configured to process at least a portion of the autocalibration process of the autocalibration module 120.

### 3. Method

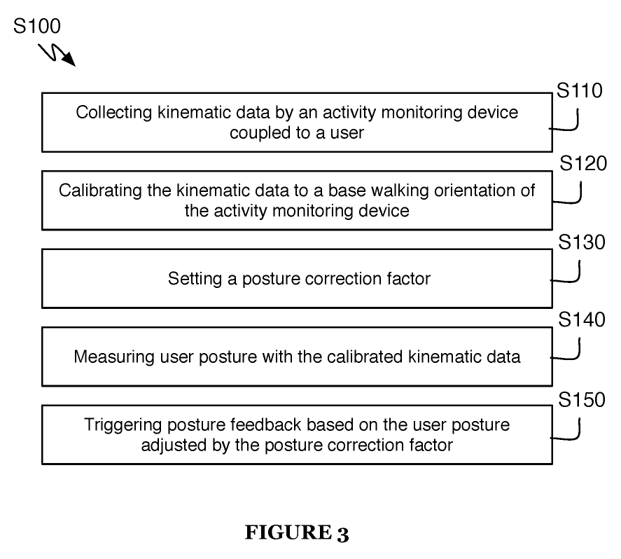

As shown in FIG. 3, a method S100 for automatic posture calibration of a preferred embodiment can include collecting kinematic data by an activity monitoring device coupled to a user S110, calibrating the kinematic data to a base walking orientation of the activity monitoring device S120, setting a posture correction factor S130, measuring user posture with the calibrated kinematic data S140, and triggering posture feedback based on the user posture adjusted by the posture correction factor S150.

The method is preferably implemented by a system such as the one described above, but may alternatively be implemented by any suitable system. The method is preferably implemented in association with at least one activity monitoring system that collects at least one point of kinematic data. For example, a sensing device may be attached to the upper chest region of a garment, but the sensing location can alternatively be any suitable location such as at the waist region, pelvic region, the back, the head, or any suitable location. Alternatively, the method can additionally involve sensing kinematic data from multiple points, and applying that kinematic data to calibrating and monitoring posture.

In one variation, the method is implemented on a standalone device connected with a sensing system. In another variation, the method is implemented on a native application operable on a personal computing device (e.g., smart phone, wearable computing device, or personal computer). In yet another variation, the method can be implemented in the cloud on a remote server. The method may alternatively be implemented through any suitable system.

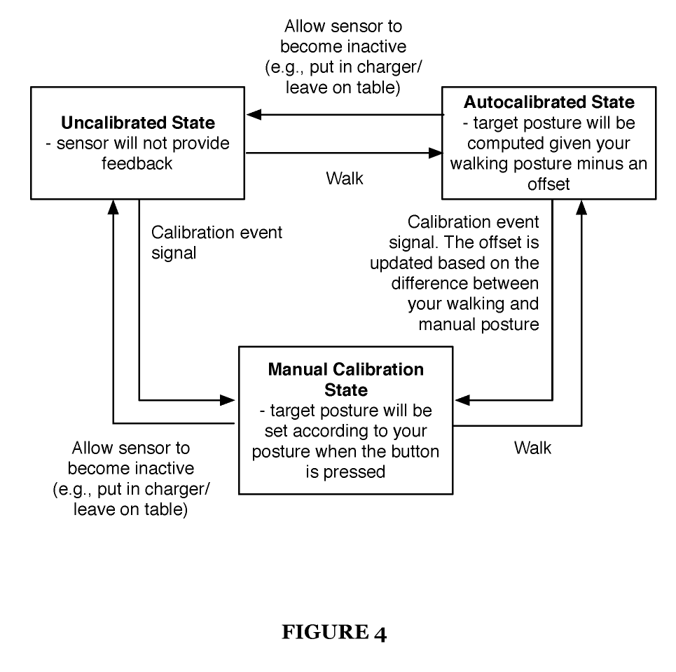

Variations of the method may use preconfigured properties in sensing and monitoring posture, user initiated calibration, and/or data-driven or machine learning. The method can additionally be used in altering operating modes of an activity monitoring device. As one example of a simple implementation, an activity monitoring device may be preconfigured with a fixed posture correction factor. In some cases, an offset of two degrees from the walking posture can approximate a good target posture for most users. In another example of state changes of an activity monitoring device, the activity monitoring device can support autocalibration in combination with manual calibration as shown in FIG. 4.

Block S110 which includes collecting kinematic data by an activity monitoring device coupled to a user functions to sense, detect, or otherwise obtain time series sensor data that reflects user motion and/orientation. In one variation, data of the kinematic data streams is raw, unprocessed sensor data as detected from an activity monitoring device. The activity monitoring device preferably includes at least one inertial measurement unit as described above, but any suitable sensing system may be used. An alternative intermediary data source could provide stored data collected from any suitable system. In another variation, the data can be pre-processed. For example, data can be filtered, error corrected, or otherwise transformed.

The individual kinematic measurements in the kinematic data preferably correspond to distinct kinematic measurements along a defined axis. The kinematic measurements are preferably along a set of orthonormal axes (e.g., an x, y, z coordinate system).

The kinematic measurements can include acceleration, velocity, displacement, force, angular velocity, angular displacement, tilt/angle, and/or any suitable metric corresponding to a kinematic property or dynamic property of an activity. Preferably, a sensing device provides acceleration as detected by an accelerometer along three orthonormal axes. The set of kinematic data streams preferably includes acceleration in any orthonormal set of axes in three-dimensional space, herein denoted as x, y, z axes. Accordingly collecting kinematic data can include collecting three-axes of accelerometer data. Additional kinematic sensor data may additionally be collected such as three-axes of angular velocity from a three-axis gyroscope. Additionally, the sensing device may detect a magnetic field through a magnetometer (e.g., three-axis magnetometer). Kinematic data is preferably collected at some sample rate (e.g., 25 Hz). When there is no movement, the accelerometer readings preferably reflect earth gravity only, resulting in:

\sqrt{x²+y²+z²}=1 g, where 1 g≅9.8 ms²

As described below, the axis of measurements may not be aligned with a preferred or assumed coordinate system of the activity. Accordingly, the axis of measurement by one or more sensor(s) may be calibrated in block S120. The relative values of x, y, and z are determined by the current orientation of the accelerometer. For calibration purposes, we want to find an orientation frame R such that when the user is standing or sitting straight in good posture:

$$R [ \begin{matrix}x \\ y \\ z\end{matrix} ] = [ \begin{matrix}0 \\ 1 \\ 0\end{matrix} ] = [ \begin{matrix}{x}^{′} \\ {y}^{′} \\ {z}^{′}\end{matrix} ]$$

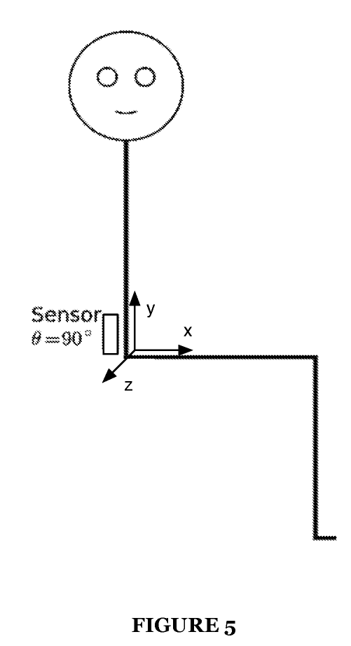

The orientation after multiplying with R should be such that x′ turns positive when bending right, and z′ turns positive when bending backwards, while y′ increases for upward acceleration for selected coordinates used herein and shown in FIG. 5. We define the forward/backward angle θ such that θ=90° for a perfectly upright position. Bending forward will result in a smaller value θ.

The activity monitoring device is preferably physically coupled to a user's body or clothing. The coupling is at least partially stable such that gross changes in relative coupling orientation/position are kept constant over short periods of time. However, the method is preferably robust enough to support localized fluctuations in relative orientation. For example, an activity monitoring device can be coupled to a shirt that is worn by a user, the movement of the shirt may change relative orientation of the activity monitoring device, but the changes are localized to changes within a region based on where on the shirt the activity monitoring device is attached. As discussed above, the activity monitoring device may be attached in a variety of locations and/or orientations. In this way a user can have more flexibility in where and how they attach the activity monitoring device. In a preferred implementation, the collecting of kinematic data streams S110 can be collected from an inconsistently mounted activity monitoring device. An activity monitoring device is generally characterized as inconsistently mounted when the orientation of a sensor is substantially not identical between different mountings (e.g., an activity monitoring device may be coupled to the user at different locations between uses) and different users. The sensor will generally have different orientations between uses and potentially during use. Block S120 of the method can preferably account for such variability in orientation changes.

Block S120, which includes calibrating the kinematic data to a base walking orientation of the activity monitoring device, functions to normalize or “center” the kinematic data for it's general orientation when walking. More generally block S120 may alternatively include calibrating the kinematic data to a base activity orientation of the activity monitoring device during the base activity. The walking activity state has particular characteristics that can make it an attractive candidate for a base activity used in calibration. Walking is commonly performed offering several opportunities to calibrate and update calibration. Walking can be detected even before an initial calibration. User posture is generally consistent and near a good posture when walking. Alternative activity states could alternatively or additionally be used. In one variation, multiple activity states may be used wherein the system can switch between calibration based on different activities.

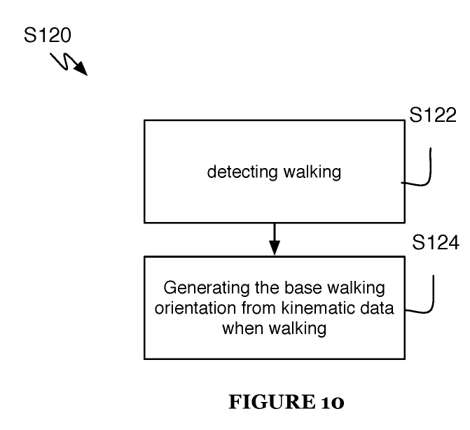

Calibrating the kinematic data to a base walking orientation preferably includes detecting walking S122 and generating the base walking orientation from kinematic data when walking S124 as shown in FIG. 10.

Block S122, which includes detecting a walking activity state through the kinematic data, functions to detect a walking activity state. Various approaches of detecting walking through kinematic data may be employed such as approaches for walk detection detected in U.S. Pat. No. 9,128,521, issued on 5 Sep. 2015, which is hereby incorporated in its entirety by this reference. The detection of a walking activity state may be performed prior to any calibration to correct for sensor position. Thus detecting a walking activity state should be robust to working under different orientations.

One potential approach to detecting walking activity state is to assess energy of accelerometer readings and compare the energy against a threshold indicative of walking. A measure of a preferable accelerometer energy score Aₜ for a sample data recorded at time t can be given by:

Aₜ=ln(xₜ²+yₜ²+zₜ²).

where xₜ, yₜ and zₜ are the accelerometer measurements along the axes x, y, and z at time t.

A change in this quantity indicates motion. Therefore, the difference to the previous frame can be computed as

Dₜ=|Aₜ−Aₜ₋₁|.

The energy score differences can be summed and compared to a threshold. In one particular implementation, the energy score differences are summed in a Bartlett window of width of two seconds. In the implementation where sampling rate is 25 Hz, this computation can be:

Sₜ=25Dₜ+Σₖ₌₁²⁴(25−k)(Dₜ₊ₖ+Dₜ₋ₓ).

And in its scaled form:

$${S}_{t} = \frac{25 {D}_{t} + {\sum }_{k = 1}^{24} ( 25 - k ) ( {D}_{t + k} + {D}_{t - k} )}{625}$$

If Sₜ, our walking score, exceeds a set threshold value at time t, then the accelerometer reading can be classified as a walking sample. In one exemplary implementation, an experimentally determined threshold of 70 (where 1G represents 2048), which is equivalent to an average 1.118G, may be used as the threshold. Such accelerometer power assessments can be performed repeatedly. A walking activity state could be detected for one such reading, but minimum number of consecutive readings may need to be classified as a walking sample to qualify as a walking activity state.

Alternative implementations may detect walking activity state through alternative sensing approaches. For example, a pedometer sensor may be used to detect when a walking cadence is detected. A walking activity state could alternatively be detected based on rate of change of location. For example, a location detection device such as a GPS or a location service of a mobile device may be used to detect changes in location. If the rate of change is within a walking speed, a walking state could be detected. In another variation, a user or other entity may signal to the activity monitor device that the user is in a walking activity state. For example, a user may hit a button indicating that the user is walking. Other alternative approaches to detecting a walking activity state may be used.

The base walking orientation is preferably established upon detecting at least a minimum amount of walking activity. In one variation, a minimum number of steps need to be detected (e.g., at least 5 steps, at least 10 steps, or the like). In another variation, the walking activity state must be detected or active for a minimum amount of time (e.g., at least 5 seconds, at least 10 seconds, or the like). The kinematic data is preferably recorded for at least that minimum amount of walking activity. Alternatively, a single snap shot of kinematic data may be used.

Block S124, which includes generating the base walking orientation from kinematic data when walking, functions to calibrate a reference orientation of the kinematic data that is to be used for posture monitoring and/or other forms of activity tracking. Various approaches for calibrating a base reference orientation may be used. One preferred implementation can include correcting pitch and/or correcting yaw of the kinematic data. Calibration of a base walking orientation can rely on the generation of one or more rotation matrices that are set to calibrate the kinematic data to that reference orientation. Preferably the base walking orientation is established through the calculation of a base orientation frame that is the result of applying the rotation matrix used in calibrating the kinematic data to a walking orientation frame. A base orientation frame may be given as R_{base} where R_{base}=R_{y}RₓR_{z}.

Correcting pitch and roll of the kinematic data can be achieved after a sufficient number of walking readings have been collected. In one variation, at least three seconds or three steps may be used. In some preferred implementations, ten steps, ten seconds or around 250 samples may be used as minimum thresholds, but any suitable threshold may be used. Natural walking may induce the spine into a good or at least consistent posture.

$$[ \begin{matrix}{x}_{0} \\ {y}_{0} \\ {z}_{0}\end{matrix} ] = [ \begin{matrix}{\overline{x}}_{walk} \\ {\overline{y}}_{walk} \\ {\overline{z}}_{walk}\end{matrix} ]$$

In one preferred implementation, a rotation matrix Rₒ can then be computed such that

$$R [ \begin{matrix}{x}_{0} \\ {y}_{0} \\ {z}_{0}\end{matrix} ] = [ \begin{matrix}0 \\ 1 \\ 0\end{matrix} ]$$

Rₒ is the product of two rotations

Rₒ=RₓR_{z}

where Rₓ is given by

$${R}_{x} = [ \begin{matrix}1 & 0 & 0 \\ 0 & cos θ & - sin θ \\ 0 & sin θ & cos θ\end{matrix} ]$$

and R_{z} by

$${R}_{z} = [ \begin{matrix}cos φ & - sin φ & 0 \\ sin φ & cos φ & 0 \\ 0 & 0 & 1\end{matrix} ] .$$

The rotation matrix Rₒ and its components Rₓ and R_{z} are used in calibrating pitch and roll. The angles θ and φ are defined as

$$θ = - sgn ( {z}_{0} ) a cos ( \frac{\sqrt{{x}_{0}^{2} + {y}_{0}^{2}}}{\sqrt{{x}_{0}^{2} + {y}_{0}^{2} + {z}_{0}^{2}}} )$$

$$and$$

$$φ = sgn ( {z}_{0} ) a cos ( \frac{{y}_{0}}{\sqrt{{x}_{0}^{2} + {y}_{0}^{2}}} ) .$$

Correcting yaw of the kinematic data can similarly be addressed in various approaches. In one simple approach, activity monitoring device orientation can be assumed to either be zero degrees or 180 degrees. An orientation assumption may be used in place of higher resolution correction. An orientation assumption could also be used as a temporary solution as sufficient kinematic data is collected. Variances and covariances used in yaw correction may necessitate a number of samples (e.g., at least 750 samples or 30 seconds of walking sampled at 25 Hz). In one preferred implementation, a set of kinematic data samples can be adjusted for error through analysis of the shape of a multidimensional plotting of the kinematic data. An assumption can be made that humans are substantially left-right symmetrical such that aligning larger eigenvectors with the z axis results in a left-right symmetrical cloud of measurements. Yaw shape correction is preferably performed after correcting pitch and roll. Here, the set of kinematic data samples are represented by:

$$[ \begin{matrix}{x}_{1} \\ {y}_{1} \\ {z}_{1}\end{matrix} ] … [ \begin{matrix}{x}_{n} \\ {y}_{n} \\ {z}_{n}\end{matrix} ] .$$

With pitch and roll correction in place, a modeling assumption can be that:

Σₖ₌₁ⁿxₖ=0 and Σₖ₌₁ⁿzₖ=0.

This modeling assumption is based on rotation of (avg(x), avg(y), avg(z)) to be on the y-axis (e.g., avg(x)=0 and avg(z)=0). A modeling assumption can also be made that

$$\frac{{\sum }_{k = 1}^{n} {x}_{k}}{N} = 1$$

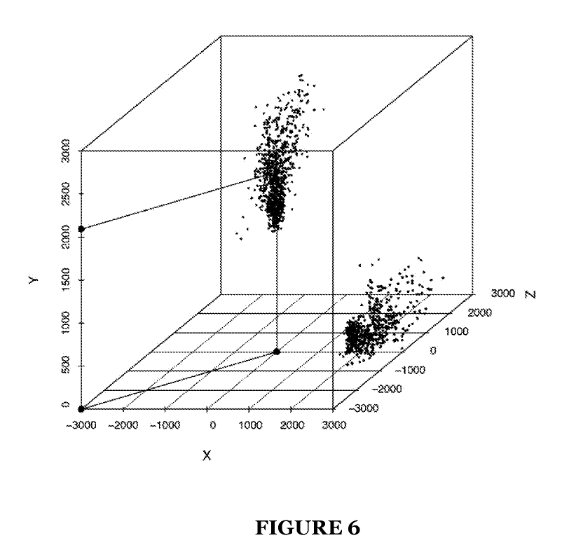

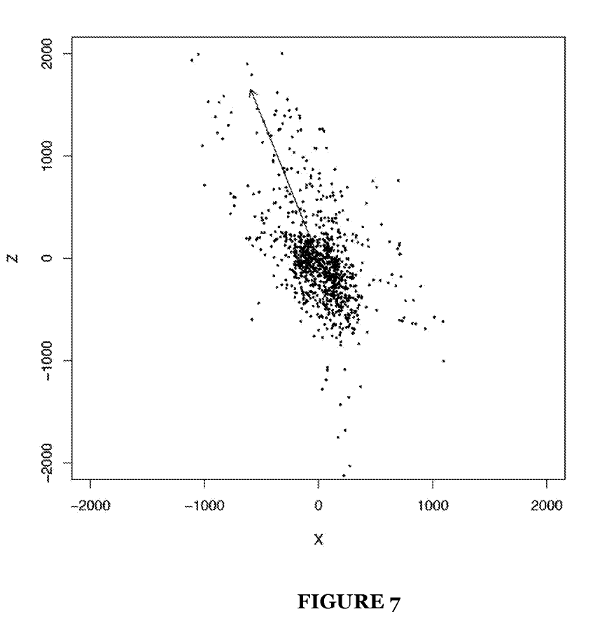

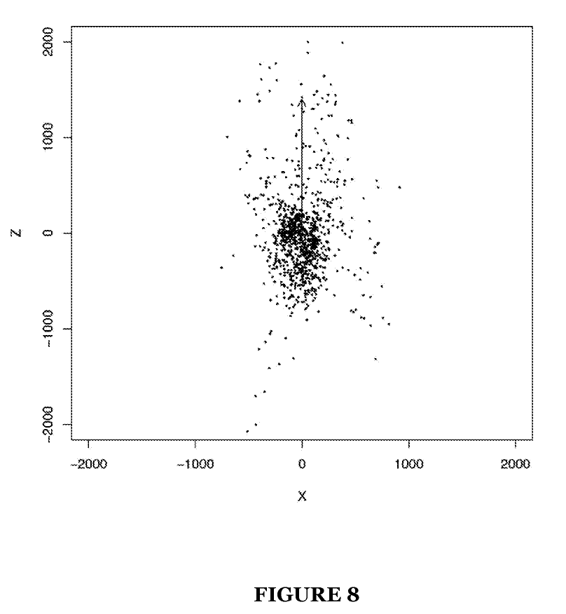

Eigenvectors of the samples can be used in determining the corrective rotation to be applied on the set of kinematic data samples. As an illustrative example, FIG. 6 shows 1000 accelerometer readings during a walking activity state before and after correcting for pitch and roll in a three dimensional plot, wherein the sensor which is worn on the lower back shifted to the right before and after correcting for pitch and roll. In this example it can be observed that the mean of x and z comes to approximately o after correction. The approximate mean of y is 1 g, which in this example is represented by the sensor value of 2048. FIG. 7 shows corresponding data in a two dimensional plot with an eigenvector, which can be used to correct for yaw. FIG. 8 shows corresponding data after rotating by an angle defined by the larger eigenvector.

Such an implementation may take advantage of Principal Component Analysis (PCA). PCA approach preferably considers only two dimensions as a simplifying strategy. Performing two-dimensional variation of PCA can include generating a covariance matrix, generating eigenvectors and eigenvalues, and correcting yaw corresponding to the angle of the eigenvector. Herein is described one particular approach for estimating covariance matrix and eigenvectors and eigenvalues that may be particularly beneficial for a computing device with limited battery, RAM, and computing capabilities. Any suitable approach may be used.

In generating a covariance matrix, an XZ covariance matrix can be defined as

$$\sum ( X , Z ) = [ \begin{matrix}var ( X ) & cov ( X , Z ) \\ cov ( X , Z ) & var ( Z )\end{matrix} ]$$

where

var(X)=E(X²)−E²(X)

and

cov(X,Z)=E(XZ)−E(X)E(Z).

Here upper case letters are used because accelerometer readings are treated as random variables. E(X) here denotes the expected value of X, which can be estimated by taking the mean of the observed readings of X or use any suitable estimation.

As the above calculations rely on averages, in one implementation, a memory saving approach can update an average with a new sample reading without storing all previous samples. Supposing a known average x̅_{N} of the previous N values of X, then:

$${\overline{x}}_{N + 1} = {\overline{x}}_{N} \frac{N}{N + 1} + {x}_{n + 1} \frac{1}{N + 1}$$

Such an averaging approach may be used to estimate E(X), E(Z), E(X²), and E(XZ), and compute an estimate of the covariance matric from the expected value estimates. Given the large sample sizes (e.g., more than 100 samples) differences from more rigorous or traditional estimators can be negligible. For the 100 points from the example shown in FIG. 6, the estimated covariance matrix is

$$\sum ( X , Z ) = [ \begin{matrix}78127 & - 98438 \\ - 98438 & 315465\end{matrix} ]$$

Generating eigenvectors and eigenvalues for dimension of two can degenerate to a quadratic equation with a closed solution. A 2D covariance matric can have the form

$$[ \begin{matrix}a & b \\ c & d\end{matrix} ]$$

Then two eigenvalues can be set to

$${λ}_{\pm } = \frac{( a + d ) \pm D}{2}$$

where

D=\sqrt{(a+d)²+4(ad−bc)}.

Since cov(X,Z)≠0, then the corresponding eigenvectors would be either

$${e}_{\pm } = [ \begin{matrix}{λ}_{\pm } - d \\ c\end{matrix} ]$$

$$or$$

$${e}_{\pm } = [ \begin{matrix}b \\ {λ}_{\pm } - a\end{matrix} ]$$

Correcting yaw corresponding to the angle of the eigenvector preferably rotates the set of kinematic data points by angle between νₑ and the z axis about the y axis, where νₑ=(xₑ, zₑ) is the eigenvector corresponding to the larger eigenvalue. The angle between νₑ and the z axis can be given by

$${φ}_{e} = a tan \frac{{x}_{e}}{{z}_{e}} .$$

Yaw correction is then a rotation around they axis by −φₑ:

$${R}_{y} = [ \begin{matrix}cos ( - {φ}_{e} ) & 0 & sin ( - {φ}_{e} ) \\ 0 & 1 & 0 \\ - sin ( - {φ}_{e} ) & 0 & cos ( - {φ}_{e} )\end{matrix} ]$$

A complete base orientation frame can then be defined as R_{base}=R_{y}RₓR_{z}. Since the negative of an eigenvector is also an eigenvector, the method can use a heuristic approach to selecting an appropriate eigenvector. If the activity monitoring device is worn on the lower back cov(Y,Z) is expected to be negative if forward/backward orientation is correct. A correction of 180 degrees can be added to the yaw correction angle if cov(Y,Z) is positive. If the sensor is worn on the front of the upper torso, the situation is reversed, and correct forward/backward orientation corresponds to a positive cov(Y,Z). Approximate sensor location may be assumed, detected, specified, or otherwise determined. Corrections for other body regions could also be used.

In the example above, φₑ computes to −19.8 degrees. As shown in FIG. 8, the set of kinematic data readings are corrected to be aligned substantially symmetrical about the x-axis, which corresponds to general left-right symmetry of a user.

Block S120 is preferably automatically triggered upon detecting the base activity state (e.g., a walking activity state). Block S120 is preferably performed upon the initial detection of the base activity state for each usage session. More preferably, Block S120 is preferably repeatedly performed such that the kinematic data can be corrected to account for changes in relative orientation of the activity monitoring device or other changes. Accordingly, block S120 includes recalibrating the kinematic data upon subsequently detecting a walking activity state, wherein the base walking orientation is updated at least partially based on a subsequent analysis of orientation.

A base walking orientation may additionally or alternatively be statically calibrated in response to a manually activated trigger. A manual calibration consists of determining the complete orientation frame, R_{target}=R_{y}RₓR_{z}, when user is in good posture and triggers a manual calibration event. A manual calibration event can be triggered by enabling a physical or virtual button on the activity monitoring device or using any suitable trigger. The target posture is then used to calibrate the orientation of the sensor, which can be subsequently used to assess the posture and biomechanics of a user. A manually calibrated targeted posture can be used as a reference posture to determine the posture correction factor in conjunction with the activity based calibration in Block S120. The manual calibration can be done either independently, as the sole method of calibration, or in combination with auto-calibration. In one variation, manual calibration may be used prior to auto-calibration activation such as before a user has walked. For example, an activity monitoring device when first activated may use a manual calibration mode until the user walks, which would activate use of an auto-calibration mode. In another variation, an activity monitoring device may include selectable calibration modes such that a user can change the calibration mode between an automatic calibration mode and a manual calibration mode. In another variation, the method may not include activity detection, and the user is directed to manually calibrate during a base activity. For example, a user may manually trigger calibration while walking or during another suitable reference activity. When a manual calibration mode is active, a posture correction factor may not be calculated or used because the user is assumed to exemplify good posture when triggering the manual calibration event. Block S130, which includes setting a posture correction factor, functions to establish a difference between the orientation at a base activity (e.g., walking) and at least a second activity (e.g., sitting). The posture correction factor preferably characterizes a generally observed difference or offset that can be used to transform posture assessments. In one implementation, a posture correction factor for a particular activity can be one or more angular offsets between a target posture orientation and a base orientation. Additionally, different activities may have different postures targets and different corresponding posture correction factors. Walking activity can be used as the reference orientation because people generally have a consistent walking posture and are generally in good posture and balanced when walking, otherwise the person would fall down. A posture correction factor can be a set of offsets from the orientation as detected by an activity monitoring device when calibrated to the walking posture. For example, a user may walk with a posture with a two degree angle forward. Since the activity monitoring device is calibrated to the walking orientation, an auto-calibrated angle with upright sitting posture (i.e., zero angle) can be detected by using the walking posture with a correction offset of negative two degrees. In this example, the posture correction factor would have been set to a negative two degree offset.

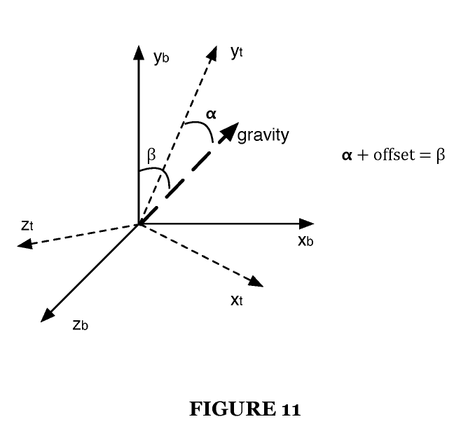

A posture correction factor can be set through a number of alternative approaches. In one variation, posture correction factors can be set by default. For example, various testing may be used to determine generally applicable posture correction factors that can be used for most users. In another variation, posture correction factors can be set based on personalized characteristics. For example, posture correction factors may be assigned to a user based on demographics (e.g., age, sex, location), fitness metrics (e.g., fitness level based on running stats), or any suitable metric. More preferably posture correction factors can be set through calibration events. A target orientation frame, R_{target}=R_{y}RₓR_{z}, can be calculated as described above through such a calibration. The posture correction factor can then be calculated through a comparison of the target orientation frame and the base orientation frame. As one example, the posture correction factor can be based in part on the angular offset(s) of the gravity vector between the base orientation frame, represented by the orthonormal axes {x_{b}, y_{b}, z_{b}}, and the target orientation frame, represented by the orthonormal axes {xₜ, yₜ, zₜ}, as shown in FIG. 11. Since the gravity vector is different in each orientation frame, the posture correction factor accounts for this difference between these frames. Heuristics and/or machine intelligence can be applied to updating a target orientation frame based on a newly calculated orientation frame for a new calibration event.

In some variations or alternative embodiments, such as when in a manual calibration mode, a targeted posture may be equivalent to the base posture where R_{base}=R_{target}, such that there is no offset component of a posture correction factor that needs to be applied. A manually set target posture is one case where there is no need for such an offset component in a posture correction factor. When the posture of the base activity state is substantially equivalent to a second activity state may be another scenario.

In some cases, different posture correction factors may be used based on the available data. For example, an activity monitoring device may initially default to a general offset, then use a posture correction factor once demographic information is received, and then use calibrated posture correction factors once the posture correction factor can be calibrated to target the user.

As mentioned, one variation of setting a posture correction factor can include setting a posture correction factor based on at least one calibration event. Block S130 can include receiving a calibration event signal through the activity monitoring device, measuring representative posture over a sustained period in response to the calibration event signal, and setting the posture correction factor based on the representative posture. The calibration event signal can be a logical signal that is triggered in response to a user interaction. For example, a user can press a calibration button on the activity monitoring device to trigger a calibration event. A user could alternatively trigger a calibration event through a connected application (e.g., using a smart phone app). The calibration event could alternatively be triggered through other mechanisms.

Measuring representative posture can include recording the orientation of the activity monitoring device for some duration. In activities where motion is present (e.g., running or walking), motion, orientation changes, and other kinematic artifacts can be processed to characterize a representative posture and offset. In some cases, the representative posture is an orientation that can be used in characterizing an offset. In other cases, the representative posture can characterize other aspects such as median posture, average posture, posture range and variation, and/or other properties of posture.

In a manual setting variation, setting the posture correction factor based on the representative posture can include replacing the posture correction factor with an updated posture correction factor that corresponds to the representative posture of calibration event. For example, a user can set a new targeted posture by sitting in a targeted posture and activating a calibration input—the previous targeted posture can be replaced or updated. More preferably, setting the posture correction factor comprises setting the posture correction factor based on processing of the representative posture and a history of representative postures from earlier calibration events. For example, the ten most recent representative postures may be averaged in setting the posture correction factor.

In a machine intelligence variation, setting the posture correction factor based on the representative posture can include setting the posture correction factor as a machine learning analysis of representative posture during multiple calibration events.

In one implementation, a large number of manually set posture correction factors can be collected and analyzed as a supervised regression problem where training offsets and target offsets can be fed through a machine learning approach, such as neural network or support vector regression, to obtain a better prediction. The posture correction factors used in the analysis may be collected for a single user, but could also be measured for a group of users.

Heuristics and/or machine intelligence can additionally be applied to detecting and addressing particular scenarios. The method can include contextually differentiating activation of a calibration input and selectively triggering either a calibration event, a silencing event, or any suitable type of event based on a scenario classification. In some scenarios, activation of the calibration input may be ignored as the current conditions do not qualify the event to be used for calibration or as a signal for silencing feedback.

One edge case scenario is to address accidental calibration events, which may occur when the button is bumped by accident. The method can include classifying the calibration event and rejecting calibration events classified as false calibrations. For example, a classifier can be set to automatically detect and reject false calibration events by looking for details in the calibration that do not fit with true calibrations. This can be treated as a supervised classification problem which may utilize neural networks, radial basis functions, support vector machines, k-nearest neighbors, and the like. Accidental calibration events could additionally or alternatively be detected through heuristics-based rules. Calibration events may be rejected and/or the updated posture correction factor may be weighted differently based on various rules. Some exemplary rules may include: detecting if the difference of the current posture correction factor differs from a newly measured posture correction factor (i.e., the one measured in response to the calibration event) is greater than a difference threshold; detecting a calibration event when prior motion was greater than a motion threshold; and detecting a change in posture correction factor greater than a change threshold. Other suitable heuristics-based rules may be used.

Another edge case scenario is when users attempt to calibrate a posture to a temporary un-ideal posture such as when they are leaning back and relaxing in a chair. The method can include classifying calibration event and suspending posture feedback during a posture state where a calibration event is classified as a silencing event. A silencing calibration event may be detected because of highly irregular posture. For example, if the calculated offset for a calibration event is greater than a set threshold, the calibration event may be considered a silencing event. For example, when a user leans far back they can activate the calibration input to “silence” posture feedback while they are relaxing. Suspension of posture feedback may last a set amount of time, until some activity condition is detected in the kinematic data, or based on any suitable condition.

The posture correction factor is preferably set for a particular activity. In particular, posture correction factors are preferably set for the sitting state. Accordingly, the method can include detecting an activity state transition between two states. In the case of monitoring posture when sitting, the method can include detecting an activity state transition between a sitting activity state and at least a second activity state (e.g., walking, running, standing, driving, etc.).

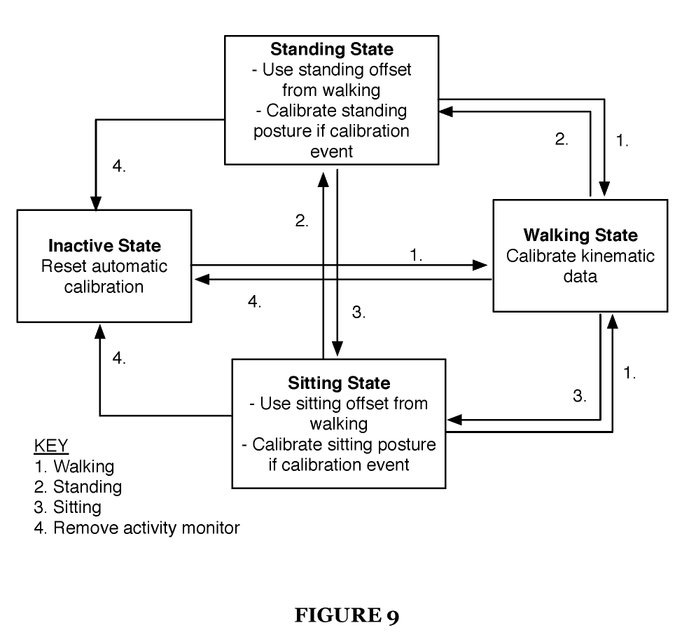

The method could additionally support multiple independent posture correction factors as shown in FIG. 9. For example, the method may enable different posture correction factors to be set and calibrated for standing, sitting, running, driving, or any suitable activity. Further posture correction factors could be assigned based on geolocation, time of day, or other suitable properties. When working with multiple posture correction factors, the method may include setting at least a second posture correction factor, wherein the first posture correction factor is for a first activity state and the second posture correction factor is for a second activity distinct from the first activity state; and wherein triggering posture feedback comprises triggering posture feedback based on the user posture adjusted by the first posture correction factor when in a first activity state and triggering posture feedback based on the user posture adjusted by the second posture correction factor when in the second activity state. The method preferably includes detecting activity state and appropriately selecting a posture correction factor.

Block S140, which includes measuring user posture with the calibrated kinematic data, functions to monitor user posture. The user posture is preferably characterized by kinematic data measurements from the activity monitoring system. The measured user posture is preferably used in assessing or tracking properties of the user's posture in block S150. Measuring user posture can be performed continuously during active states of the activity monitoring device. For example, measuring user posture may alternatively be limited to particular activity states. For example, user posture may only be performed when the user is detected to be in a sitting activity state. Measuring user posture may include generating a current orientation frame, which may be used in comparing to a base orientation frame and a posture correction factor in block S150.

Measuring user posture with the calibrated kinematic data can additionally include assessing the quality of the user posture, which functions to judge posture after accounting for calibrated orientation. Measuring user posture and, more specifically, assessing the quality in one implementation can include collecting kinematic data in a similar manner as described above and generating a comparison to the base orientation adjusted by the posture correction factor. The comparison may be approached in a variety of ways. In one implementation, a current orientation frame can be calculated in a substantially similar manner to the base orientation frame above, where the current orientation frame is based on the recently sampled kinematic data. The current orientation frame, R_{current}, can then be compared to the base orientation frame, R_{base}, corrected or augmented by the posture correction factor. R_{target} may not need to be updated as often throughout the day if the sensor orientation shifts because the R_{base} orientation frame is constantly or periodically updated. In another implementation, real-time kinematic time series data is transformed by R_{base} into a real-time posture value and compared to an ideal posture as defined by R_{base} and the posture correction factor (e.g., a posture offset). This comparison can indicate an orientation difference, which can be a measure of the real-time posture value's deviation from a targeted ideal posture similar to an offset. For example, a user leaning five degrees forward from an ideal sitting posture may have an orientation difference of five degrees. The orientation difference can additionally characterize posture deviations along various vectors (e.g., backward angle orientation difference, leaning to the side orientation difference, and the like). The orientation difference can then be analyzed against various conditions as a way of assessing the posture quality. Preferably, this orientation difference can be compared to a set of posture thresholds that can characterize different types of posture such as characterizing “great posture”, “normal posture”, and/or “bad posture”. A posture threshold may be defined as an angular range of orientation about one or more axes, but any suitable characterization may be used. For example, a good posture could be characterized as an angular range centered (or containing) an ideal posture angle, and a bad posture could be characterized as user posture with an angle outside of the angular range. The ideal posture is generally the orientation frame that the posture correction factor is promoting as the targeted posture. Alternative posture conditions could account for other factors such as the mean user posture over time, total duration in a particular posture over the course of a day or time window, changes in posture, and/or other factors. In an additional variation, posture conditions may change based on the training of the posture correction factor. For instance, the variation allowed for a target posture can adjust based on the variation in training samples.

Block S150, which includes triggering posture feedback based on the user posture adjusted by the posture correction factor, functions to react to the current user posture when judged against a targeted posture state. Posture feedback is preferably delivered when the current posture satisfies a condition. Posture feedback can be positive—indicating that the user is demonstrating good posture. Posture feedback can additionally, or alternatively, be negative—indicating that use is not demonstrating good posture. Heuristics-based rules and/or algorithmic analysis may be used in determining when and how posture feedback is delivered. The assessment and analysis of posture quality from Block S140 is preferably used in determining posture feedback. For example, a set of posture conditions could be set with posture range thresholds used in determining classifying posture (e.g., great posture, ok posture, bad posture, etc.) and specified forms of feedback are delivered based on the classifications of posture. In another example, machine learning could be applied to apply feedback in a dynamic manner such that the feedback is modeled to promote better posture. This can be treated as a supervised classification problem which may utilize neural networks, radial basis functions, support vector machines, k-nearest neighbors, and the like.

Feedback could be delivered in a variety of approaches including but not limited to tactile feedback, audio feedback, graphical feedback (on device or in an application), informational feedback (data analysis representations), and/or other forms of feedback. For example, feedback can be tactile vibration feedback delivered through the activity monitoring device or an application when posture deviates from a targeted posture. The feedback could alternatively be informational and represented through a data representation on a graphical user interface or in a generated report.

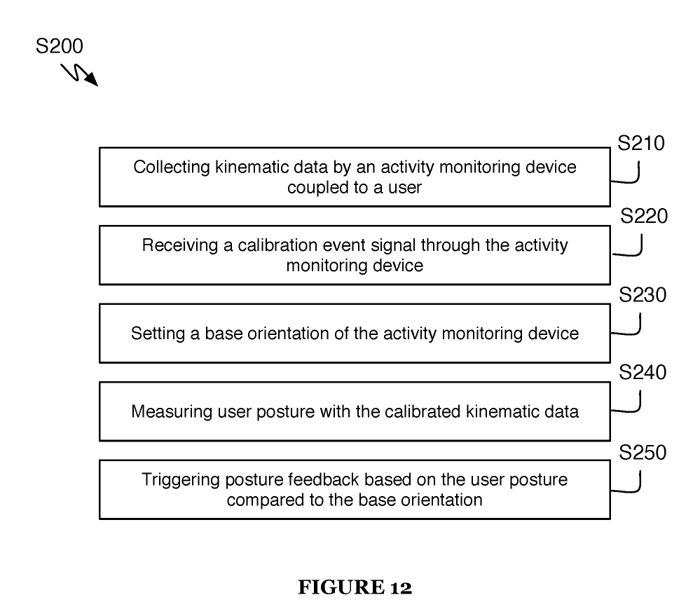

As shown in FIG. 12, a method S200 for manual calibration of one preferred embodiment may utilize some of the approaches described herein for an activity monitoring device using only manual calibration which may include collecting kinematic data by an activity monitoring device coupled to a user S210; receiving a calibration event signal through the activity monitoring device S220; in response to detecting a calibration event, setting a base orientation of the activity monitoring device S230; measuring user posture with the calibrated kinematic data S240; and triggering posture feedback based on the user posture compared to the base orientation S250. Here setting a base orientation of the activity monitoring device can be substantially similar to setting the base orientation of block S120 and updating the base orientation can be used in place of setting a posture correction offset. This method may be used independently or in combination with the autocalibration method described herein. In one mode, a method can include an activity monitoring device performing method S100 when in an autocalibration mode and performing the method S200 when in a manual mode.

Many of the various approaches used in setting the posture correction factor described above can be applied to setting the base orientation during manual calibration.

In variations where calibration events are used to set or update a base orientation, setting the orientation frame to calibrate kinematic data may utilize some of the same approaches used in setting the posture correction factor.

In one variation, a manual calibration mode may include contextually differentiating calibration events and filtering or discarding calibration events, which can function to ignore false button triggers. Similarly, the method can include altering weighting of a new orientation frame when updating the base orientation frame of the base orientation based on patterns in a calibration event.

Another variation of a manual calibration mode can include setting a base orientation as a machine learning analysis of multiple calibration events.

Another variation of a manual calibration mode can include setting different base orientations for particular activity states based on a detected activity state when the calibration event occurs. For example, different orientation frames may be set for different manually calibrated postures for sitting, standing, walking, and the like.

Yet another variation of a manual calibration mode can include classifying calibration event and suspending posture feedback during a posture state in a similar fashion as above. Other variations of the autocalibration approach can similarly be applied to the manual calibration mode.

The systems and methods of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

We claim:

1. A method for posture feedback comprising:
collecting kinematic data by an activity monitoring device coupled to a user;
calibrating the kinematic data to a base walking orientation of the activity monitoring device, which comprises:
detecting a walking activity state through the kinematic data, and
when a walking activity state is detected, generating the base walking orientation from kinematic data;
setting a posture correction factor;
measuring user posture with the calibrated kinematic data;
triggering posture feedback based on the user posture adjusted by the posture correction factor.

2. The method of claim 1, wherein setting a posture correction factor comprises receiving a calibration event signal through the activity monitoring device.

3. The method of claim 2, wherein setting a posture correction factor further comprises measuring user posture over a sustained period in response to the calibration event signal, and setting the posture correction factor as an average of measured user posture during multiple calibration events.

4. The method of claim 2, wherein setting a posture correction factor further comprises measuring user posture over a sustained period in response to the calibration event signal, and setting the posture correction factor as a result of machine learning analysis of measured posture of multiple users.

5. The method of claim 2, further comprising classifying the calibration event signal and rejecting calibration events classified as false calibrations.

6. The method of claim 2, further comprising classifying the calibration event signal and suspending posture feedback during a posture state where a calibration event is classified as a silencing event.

7. The method of claim 2, wherein the activity monitoring device is attached to an article of clothing of a user and the calibration event signal is triggered by the activation of an input on the activity monitoring device.

8. The method of claim 1, wherein calibrating the kinematic data to a base walking orientation comprises recalibrating the kinematic data to an updated base walking orientation upon subsequently detecting walking.

9. The method of claim 8, wherein calibrating the kinematic data to a base walking orientation further comprises recording kinematic data during the walking activity state for at least five steps.

10. The method of claim 1, further comprising detecting a sitting activity state, wherein measuring user posture with the calibrated kinematic data is measured during the sitting activity state.

11. The method of claim 1, further comprising setting at least a second posture correction factor, wherein the first posture correction factor is for a first activity state and the second posture correction factor is for a second activity distinct from the first activity state; and wherein triggering posture feedback comprises triggering posture feedback based on the user posture adjusted by the first posture correction factor when in a first activity state and triggering posture feedback based on the user posture adjusted by the second posture correction factor when in the second activity state.

12. The method of claim 1, wherein generating the base walking orientation from kinematic data comprises correcting pitch and roll of the kinematic data.

13. The method of claim 11, wherein generating the base walking orientation from kinematic data comprises correcting yaw of the kinematic data.

14. A system for posture feedback comprising:
an activity monitoring device that couples to a user that includes
an inertial measurement unit that collects kinematic data,
user feedback mechanism, and
a processor; and
wherein the processor is configured to:
detect a walking activity state through the kinematic data,
calibrate the kinematic data when in the walking activity state,
set a posture correction factor,
measure user posture, and
activate the user feedback mechanism based on the user posture adjusted by the posture correction factor.

15. The system of claim 14, wherein the activity monitoring device further comprises a calibration input; and wherein the processor is further configured to:
detect a calibration event signal triggered by the calibration input,
measure user posture over a sustained period in response to the calibration event signal, and
set the posture correction factor as an average of measured user posture during multiple calibration events.

16. The system of claim 15, wherein the calibration input is a button, and the user feedback mechanism is vibrational actuator.

17. The system of claim 14, wherein the activity monitoring device further comprises a calibration input; and wherein the processor is further configured to:
detect a calibration event signal triggered by the calibration input,
measure user posture over a sustained period in response to the calibration event signal, and
set the posture correction factor as a machine learning analysis of measured posture of multiple users.

18. The system of claim 14, wherein the processor is configured to classify the calibration event signal and suspend activation of the user feedback mechanism where a calibration event is classified as a silencing event.
